(19) United States
(12) Patent Application Publication
(10) Pub. No.: US 2010/0135976 A1
(43) Pub. Date: Jun. 3, 2010

(54) ADSORPTION DEVICE

(76) Inventors: Rune Nilsson, Lund (SE); Bengt Sandberg, Hjarup (SE)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/445,542

(22) PCT Filed: Jun. 18, 2007
(86) PCT No.: PCT/SE07/00594

§ 371 Date: Jan. 27, 2010

Related U.S. Application Data
(60) Provisional application No. 60/814,376, filed on Jun. 16, 2006.

(30) Foreign Application Priority Data
Jun. 16, 2006 (SE) 0601330-4

(51) Int. Cl. A61K 35/12; B01D 15/04; C12Q 1/02

(52) U.S. Cl. 424/93.73; 210/263; 210/206; 210/661; 435/29

(57) ABSTRACT

The invention relates to an adsorption device for removal and/or reduction of undesirable components from blood, such as whole blood, comprising, porous cross-linked polysaccharide particles or beads, wherein the particles or beads have an average size from about 210±50 μm, at least 30% of the particles have a size from about 180 to about 240 μm, at least 70% of the particles have a size from about 150 to about 300 μm and said particles have at least one immobilised ligand, wherein said immobilised ligand have a size of about 150 kD or less. The invention also relates to kits comprising said adsorption device as well as the use of said adsorption device in different applications such as cleaning of blood.

### FIELD OF INVENTION

The invention relates to specific properties of particles used in an adsorption device for removal and/or reduction of undesirable components from blood, such as whole blood, comprising porous polysaccharide particles or beads, which may be spherical and cross-linked, wherein the particles have an average size about 210±50 μm, at least 30% of the particles have a size from about 180 to about 240 μm, at least 70% of the particles have a size from about 150 to about 300 μm and said particles having at least one immobilised ligand, wherein said immobilised ligand has a size of about 150 kD or less. The invention also relates to an adsorption device containing such particles, kits comprising said adsorption device as well as the use of said adsorption device in different applications such as cleaning of blood.

### BACKGROUND OF INVENTION

Extracorporeal techniques are useful for removing unwanted and/or pathogenic substances from the blood either through an on-line system in which the blood is withdrawn from the patient, purified, and continuously returned to the patient as whole blood or plasma (Lindén et al., Cancer Biotherapy & Radiopharmaceuticals. 20(4): 457-466 (2005)) or through a discontinuous process where a limited volume of blood is collected or assembled from a mammal such as a human patient, purified from undesired and/or pathogenic substances, and returned to the patient (Freischlag J. A., Critical Care 2004, 8 (Suppl 2):S53-S56, and references therein).

Extracorporeal techniques are also useful for the clearance of medical agents from blood circulation. Applications of these methods using the technique in context of immunotherapy have previously been described (Henry Chemical Abstract 18:565 (1991); Hofheinze D. Et al., Proc. Am. Assoc. Cancer Res. 28:391 (1987); Lear J. K. Et al. Antibody Immunoconj. Radiopharm. 4:509 (1991); Dienhart D. G. Et al., Antibody Immunoconj. Radiopharm. 7:225 (1991); De Nardo et al., J. Nucl. Med. 34:1020-1027; De Nardo et al., J. Nucl. Med. 33:863-863 (1992); and U.S. Pat. No. 5,474,772 (Method of treatment with medical agents)).

To make the blood clearance more effective and to enable processing of whole blood, rather than blood plasma as the above methods refer to, the medical agents (e.g., tumour specific monoclonal antibody carrying cell killing agents or radio nuclides for tumour localisation) can be biotinylated and cleared by an avidin/streptavidin coated adsorbent. A number of publications provide data showing that this technique is both efficient and practical for the clearance of biotinylated and radionuclide labelled tumour specific antibodies (Norrgren K. et al., Antibody Immunoconj. Radiopharm. 4:54 (1991), Norrgren K et al., J. Nucl. Med. 34:448-454 (1993); Garkavij M. et al., Acta Oncologica 53:309-312 (1996); Garkavij M. et al., J. Nucl. Med. 38:895-901 (1997).

These techniques are also disclosed in EP 0 567 514 and U.S. Pat. No. 6,251,394. The device, MitraDep®, developed by Mitra Medical AB, Lund Sweden, is based on this technology. By using a filter coated with avidin in conjunction with biotin labelled therapeutic antibodies, the whole blood clearance technique can be applied equally well for chimeric as well as fully humanised antibodies or derivatives of such antibodies. Experimental data reveal that during a three hours adsorption procedure, around 95% of the circulating biotinylated antibodies can be removed by the MitraDep® system which is also what is theoretically possible (see FIG. 2).

In order to be adsorbed to the extracorporeal filter, the antibodies carrying the cytotoxic agent (e.g., radionuclide) need to be biotinylated (biotin binds irreversible to the avidin in the filter) prior to administration to the mammal. A further development of this method with simultaneous labelling of biotin and cytotoxic agents such as radionuclides is disclosed in the patent application WO 00/02050, wherein a trifunctional reagent for the conjugation to a biomolecule is described. To facilitate the labelling of the naked therapeutic or diagnostic antibody and to ensure that the ratio of biotin and the radiolabel is one to one, Mitra Medical AB, Lund, Sweden has developed a series of novel water soluble structures (Tag-reagent, MitraTag™),

However, to fully utilize the method of clearing excess components from the whole blood, the adsorption device must be optimized with respect to a number of parameters of which some are disclosed in WO 01/95857. The average size of the particles as well as the size distribution are two of these crucial factors. It is important that the extracorporeal device comprises particles with a rather narrow size distribution to permit the blood cells in the whole blood to pass through the adsorption device without getting trapped between the particles. Hence, the void volume between the particles or beads must be sufficient to avoid such trapping. Moreover, the extracorporeal device should enable the blood cells, such as erythrocytes, thrombocytes as well as the leukocytes to pass the device and be returned back into the mammal without any significant loss of blood cells. If not, the mammal needs to be compensated for the loss of such components, which may be complicated and under certain circumstances or even impossible, such as when the mammal has a rare blood group. The infection risk as well as the risk associated with transmission of infectious agents, which may occur during transfusion needs also to be considered. Additionally, it is important that the blood cells are intact and not activated or aggregated during or after passing through the extracorporeal device. Physical stress to erythrocytes could easily lead to haemolysis and activation of thrombocytes could lead to aggregation and hemaglutination. These parameters have been carefully evaluated on agarose particles forming the bases for this invention (Bosch, T et al., 2000 Artificial Organs 24(9):696-704).

On the other hand, the clearance of the component from the blood should be performed in such a way that the treatment is as short as possible without harming the mammal or the blood to be treated.

This can be achieved by using the smallest possible size of the particles and thereby limiting the diffusion distance in the void volume. Hence, even at a higher linear flow-rate (>3 cm/min), the residence time in the adsorbent bed should be sufficient for the undesired blood components to diffuse to, and interact with, the interior of the coated particles.

A third factor is the binding capacity of the device. If only the coated surface of the particles is utilized for adsorption of the undesired blood components the device will have a very limited adsorption capacity. Hence, the porosity of the particles will be vital for the practical use of such an adsorbent. With a suitable porosity the capacity of the adsorbent can be increased 100-1 000 folds. Today, there is no adsorption device available on the market, which fulfils the above defined criteria. Therefore, there is a need for developing such an adsorption device to enable the possibility to remove undesirable components from blood, such as whole blood, without influencing other components in the blood, which are not to be removed.

### SUMMARY OF THE INVENTION

The invention relates to improved polysaccharide particles or beads with specific characteristics as part of an adsorption device for the removal of at least one component from blood, such as whole blood, from a mammal such as a human being. Said device comprises porous polysaccharide particles, which may be spherical and cross-linked, wherein the particles have an average size about 210±50 μM, at least 30% of the particles have a size from about 180 to about 240 μm and at least 70% of the particles have a size from about 150 to about 300 μm and said particles or beads having at least one immobilised ligand, wherein said immobilised ligand has a size of about 150 kD or less.

In one aspect, the invention relates to a product, the use of such product, and a kit comprising such product, comprising the above defined particles or beads having at least one ligand immobilized directly to the particles or beads or immobilized through a suitable linker. In a further aspect the invention relates to a method of using such product for the depletion of at least one “component” and/or at least one “targeting agent” from mammalian blood.

In another aspect, the invention relates to a product, the use of such product, and a kit comprising such product, comprising the above defined particles having at least one immobilized ligand (e.g. biotin binding molecule such as. avidin or streptavidin) to which a “target binding moiety” is linked to the ligand via a linker and/or spacer to a biotin or a biotin derivative. In a further aspect the invention relates to a method of using such product for the depletion of at least one “component” and/or at least one “targeting agent” from mammalian blood.

In yet another aspect the invention relates to a kit for removal of component from blood comprising an adsorption device containing the particles or bead with an immobilized ligand defined above and a container (e.g. infusion bag) comprising at least a pharmaceutically acceptable agent and at least a first biotin molecule coupled via a linker to a target binding moiety. In one embodiment, said linker is omitted.

In a further aspect, the invention relates to a kit for removal of component from blood comprising an adsorption device and a conjugate, said conjugate comprises a trifunctional cross-linking moiety selected from the group consisting of triaminobenzene, tricarboxybenzene, dicarboxyaniline and diaminobenzoic acid, said trifunctional cross-linking moiety being coupled a) via a linker 1 to a biotin molecule selected from the group consisting of biotin derivatives having essentially the same binding function to avidin or streptavidin as biotin, b) via a linker 2 to a biotin molecule selected from the group consisting of biotin derivatives having essentially the same binding function to avidin or streptavidin as biotin, and c) via a linker 3, to a target binding moiety wherein said linkers contains hydrogen bonding atoms (e.g. ether or thioether residues).

In a further aspect the invention relates to a kit for removal of targeting agent from blood comprising an adsorption device and a conjugate, said conjugate comprising a targeting agent/molecule bound via a linker 3 to at least one trifunctional cross-linking moiety selected from the group consisting of triaminobenzene, tricarboxybenzene, dicarboxyaniline and diaminobenzoic acid, said trifunctional cross-linking moiety being coupled a) via a linker 1 to a biotin molecule selected from the group consisting of biotin derivatives having essentially the same binding function to avidin or streptavidin as biotin, b) via a linker 2 to at least one cytotoxic agent; wherein said linkers contains hydrogen bonding atoms (e.g. ether or thioether residues).

In a further aspect the invention relates to a kit for removal of component from blood comprising an adsorption device as defined above and a conjugate in which the conjugate comprises multiple cytotoxic agents, whereby said conjugate has an increased cytotoxic activity.

A further aspect of the invention relates to conjugates comprising more than one trifunctional crosslinking moiety per targeting agent and where each such trifunctional crosslinking agent is coupled a) via linker 1 to a biotin molecule defined as above b) via linker 2 to one or more cytotoxic agents and c) via linker 3, to a targeting agent/molecule.

In still a further aspect, the invention relates to the use of the adsorption device or the kit for the removal and/or reduction of targeting agent/molecule and/or component from blood.

In a further aspect, the invention relates to the use of an adsorption device containing these specific particles or bead or a kit comprising such adsorption device for the removal and/or reduction of at least one undesirable component and/or targeting agent from the blood of a mammalians, such as humans. The invention also relates to a method for removing such undesirable component or components.

Finally the invention relates to the use of the adsorption device or the kit of the invention for the treatment of a disease or diagnosis of a disease such as cancer.

By providing such an improved adsorption device as well as kit's comprising said adsorption device it is possible to remove and/or reduce specific components such as toxic components from blood without influencing the other cell components of the blood. Additionally the adsorption device enables the possibility to remove the toxic components from the blood in a fast and efficient way without harming the different cells within the blood.

Further advantages and objects with the present invention will be described in more detail, inter alia with reference to the accompanying drawings.

### BRIEF DESCRIPTION OF THE DRAWINGS

FIG. 1 shows the system used in example 3 consisting of a sampling (1), a blood reservoir with mixing (2), a pump (3), a pressure monitor (4), an air detector (5) and an avidin-agarose column (6).

FIG. 2 shows the number of volumes to be adsorbed at different clearance in order to remove 95% of the substance.

FIG. 3 shows the influence of bead size on clearance.

FIG. 4 shows haemolysis of the blood during adsorption to the adsorption device.

### DETAILED DESCRIPTION OF THE INVENTION

### Definitions

In the context of the present application and invention, the following definitions apply:

The term “adsorption device” is intended to mean a device, which is capable of adsorbing components as defined below from blood such as whole blood and thereby removing and/or reducing the amount of the component in the blood. One example of such a device is an extracorporeal device. The adsorption device may be used to purify components from blood as well as removing components from blood such as mammalian blood, which needs to be cleaned prior to being returned to said mammal.

The term “component” is intended to mean any component present in the blood, which needs to be removed and/or reduced, such as a material which can harm a mammal, such as cells, substances and molecules. Examples are tumour cells, allergens, drugs, toxins such as endotoxin or other pyrogens such as those from microorganisms as well as micro-organisms, proteins and parts thereof. Other examples are targeting agents/molecules, with or without a toxic payload, where such payload could constitute a cytotoxic agent. Other components are molecules or cells, which, are normally present in the blood but which needs to be removed and/or purified from the blood under special circumstances such as incompatibility with foreign tissues or cells or molecules. The “component” can be endogeneous or exogeneous. In the latter case, the component may be administered to the mammalian at a certain time prior to the depletion from the blood (e.g. targeting agents/molecules).

The term “polysacchride” refer to polymers made up of monosaccharides joined together by glycosidic linkages. They may be branched or unbranched. One example of a polysaccharide is agarose, a polysaccharide comprising D-Galactose and 3,6-anhydrogalactose

The term “polysaccharide particles” are intended to mean particles or beads, in particular spherical beads, made of, or comprising, polysaccharide material or particles or beads coated with polysaccharide material. Agarose particles or beads are in this invention defined as particles or beads partly or completely made of agarose or are particles or beads coated with agarose. The agarose may be obtained from algae, such as seaweed or made synthetically.

The term “settled gel” is intended to mean the lower phase of a slurry of agarose gel in a water containing solution which has been settled and thereby forming an essentially agarose particle free upper phase. In this invention “ml settled gel” of an agarose gel slurry is determined by allowing such slurry to settle in a measuring cylinder for 6 hours.

The term “ligand” is intended to mean any molecule having a size of about 150 000 kD or less, which may be immobilized, with or without a linker, to said polysaccharide particles, wherein said immobilised ligand facilitate the adsorption of components which are to be removed from blood.

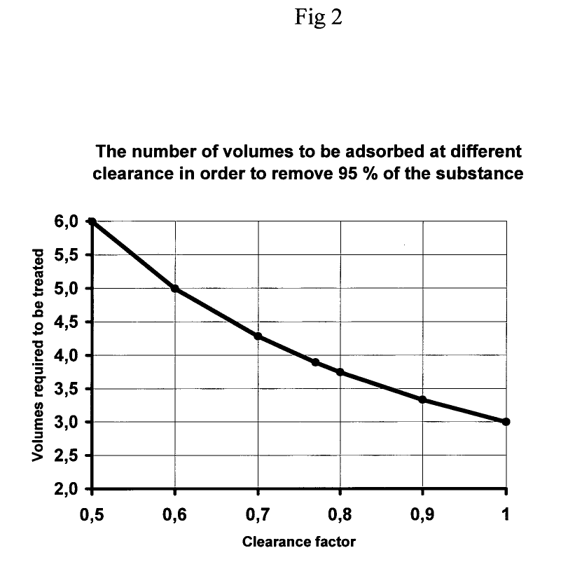

The term “clearance factor” is intended to mean the efficacy of depletion expressed as ml depleted per ml processed. The clearance factor is influenced by the flow rate, the viscosity of the solution passing through the device as well as the dimensions of the device. The clearance factor is determined by analysis of the change in IgG levels during re-circulation of an IgG containing solution through the invented adsorption device (FIG. 2).

The term “compression factor” is intended to mean the difference between bed height of particles in an adsorption device prior to being exposed to the flow-rate test in example 4 and the bed height determined after the flow rate test. The difference in bed height which occurs when the adsorption device is used in the flow rate test is denoted “compression factor”.

The term “biotin molecule” is intended to mean a biotin molecule selected from the group consisting of biotin and biotin derivatives, such as norbiotin, homobiotin, diaminobiotin, biotin sulfoxide, biotin sulfone or other biotin molecules having the ability to bind to and having essentially the same binding function to avidin or streptavidin as biotin such as having an affinity constant of ≧10⁶, such as 10⁷, 10⁸, 10⁹, 10¹⁰, 10¹¹, 10¹², 10¹³, 10¹⁴, 10¹⁵.

The term “target binding moiety” is intended to mean any moiety which can be immobilized to a matrix such as polysaccharide particles or beads, optionally through a linker, to a ligand and where said ligand is immobilized to polysaccharide particles or beads and where said “target binding moiety” having the ability to interacting with one or several types of “components” to be removed and/or reduced from liquid such as, but not limited to, blood or blood plasma or blood serum or peritoneal liquid or cerebrospinal liquid. An example of this is a target binding moiety linked to a biotin group via a “linker” and where said biotin group can be further linked to an avidin molecule immobilized to polysaccharide particles or beads.

The term “targeting agent/molecule” is intended to mean any agent that is capable of specifically targeting to specific target molecules, cells or tissues present in the blood or cells or tissues of a mammal such as a human being. A typical targeting agent/molecule is administered to the mammal, such as a human being, at a certain time period prior to the removal/reduction of said targeting agent/molecule from the blood of the mammal. Examples of targeting agents/molecules are antibodies, such as monoclonal antibodies, vitamins, such as vitamin D or folic acid and derivatives thereof, lipids, lipoproteins, carbohydrates, hormones, neurotransmitters, proteins and peptides and parts thereof, synthetic and semisynthetic variants thereof. The antibody can also be an antibody fragment such as F(ab′)₂, F(ab′), 2Fab′, F/ab), genetically engineered hybrids such as a humanised or a chimeric antibody or chemically synthesised peptides. Examples of antibodies are antibodies against different cancers mentioned in the application such as HMFG1, hMN14, trastuzumab (Herceptin®), pertuzumab (Omntarg®), BR96 ibrutomab, Erbitux®, Mylotarg® Zevalin®, LymphoCide, MabCampath®, Oncolym®, vitaxin, Avastin® rituximab and tositumomab. The term “targeting agent/molecule” also includes molecules which have been designed to have properties similar to immunoglobulin but which are derived from, or evolved from, scaffolds other than the immunoglobulin structure, such as, but not limited to, Anticalins which are based on the lipocalin core structure and “Affibody” based on the protein A structure. Dimers, trimers or multimers of such non-immunoglobulin based scaffolds are also included in this group. Typically, “targeting agents/molecules” as defined above, are interacting selectively with one or more targets in mammals such as human beings.

The term “cytotoxic agent” is intended to mean an agent, which actively reduce the number of cells and/or eliminates the cells that the targeting agent is directed against. Said cytotoxic agent may be any kind of compound as long as the compound reduces the number of target cells and or eliminate the target cells to be targeted. Said cytotoxic agent may be a natural or synthetic agent acting at different mechanism such as inhibiting DNA or RNA synthesis, inhibiting protein synthesis or interaction with tubulin, topoisomerase inhibitors, ionophores and interaction with heat shock proteins. Examples of cytotoxic agents are taxanes, such as Taxotere® and Taxol®, geldanamycin, ricin, abrin, diphtheria toxin, modecin, tetanus toxin, mycotoxins, mellitin, α-amanitin, pokeweed antiviral protein, ribosome inhibiting proteins, alkylating agents such as Auristatins, duocarmycin actinomycin, ansamitocin-P3, duocarmycin, duocarmycin B2, maytansine, maytensinoids (DM1, DM2, DM3, DM4), calicheamicin, echiomycin, 1-hydroxyauramycin A, aclacinomycin A, bafilomycin C1, dinaktin, doxorubicin, geldanamycin, leptomycin B, pluramycins, staurosporine, nogalamycin, rhodomycins, mithramycin, rabelomycin, rapamycin, alnumycin, chartreusin, geliomycin, gilvocarcin, piericidin, chlorambucil, cyclophosphamide, melphalan, and cyclopropane and antimetablites such as methatrexate, dichlorormethatrexate, cisplatin, carbopltin and metallopeptides containing platinum, copper, vanadium, iron, cobolt, gold, cadmium, gallium, iron zinc and nickel or radionuclides, such as α, β or gamma-emitters. However, the cytotoxic agent may be any agent that has the characteristics defined above and well-known for a person skilled in the art.

The term “linker” is intended to mean any structure, which will link and immobilize a “ligand” and/or “target binding moiety” to a matrix either directly or through another immobilized molecule. The “linker” could also serve as a “spacer” to prevent sterical hinderence and/or introduce extended water solubility. A “biotin-linker” is intended to mean any linker structure which could immobilize a “target binding moiety” to avidin/straptavidin, and where said avidin/streptavidin is immobilized to a matrix, such as polysaccharide particles or beads. The “linker” and the “biotin-linker” could be of various length and contain moieties promoting increase water solubility such as, but not limited to, amino-, amido-, carboxyl-, sulphate-, ether or thioether groups. The “linker” and the “biotin-linker” could be attached to the “target binding moiety” through the use of different functional groups available such as amino groups, carboxyl groups, sulfhydryl groups and carbohydrate groups. Any skilled in the art would find means of accomplish this and various methods are presented in Avidin-Biotin Chemistry: A Handbook, Pierce, 2005.

The term “Pharmaceutically acceptable agent” is intended to mean an agent such as a carrier, diluent, stabilisers or excipient that at the dosage and concentrations employed does not cause any unwanted effects to the blood components or in the patient to which it is administered. Such pharmaceutically acceptable agents are well known in the art and may be found in Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000).

### An Adsorption Device

In a first embodiment, the invention relates to an adsorption device for removal and/or reduction of components from blood, such as whole blood. The adsorption device comprises porous polysaccharide particles, preferably spherical and cross-linked beads, wherein the particles have an average size about 210±50 μm, such as about 210±40 μm or 210±30 μm. At least 30% of the particles have a size from about 180 to about 240 μm, such as 40 or 50% of the particles have a size from about 180 to about 240 μm and at least 70% of the particles have a size from about 150 to about 300 μm, such as 80 or 90% of the particles have a size from about 150 to about 300 μm. The size of the particles provides an improved device allowing all the cells as well as other components in the blood to pass through the device at a rate, which allow substantially all the components, which are to be removed and/or reduced to be efficiently removed and/or reduced. Additionally, the blood cells such as erythrocytes, leucocytes or thrombocytes or other essential components of whole blood, which are not to be removed and/or reduced should not be effected by passing the device. The effect on the cells passing through, or have passed through the device, may be studied by various methods, e.g. the blood cells counts may be determined by the Coulter method Coulter (Coulter Counter STKS, Beckman Coulter, GmbH, Krefelt, Germany). Thrombocyte activation may be determined by ELISA of β-thromboglobulin (β-TG) (Asserachrom β-TG, Boeringer Mannheim, Germany). Interleukin-1 production, which is an indication of monocyte activation may be determined by ELISA (Predicta Genzyme Diagnostics, Cambridge, Mass., USA), coagulation cascade activation may be determined by thrombin-antithrombin (TAT) complex formation (Enzygnost TAT micro ELISA by DADE Behring, Marburg, Germany) and complement activation may be determined as C3a-desArg ELISA (Progen Biotechnik, Heidelberg, Germany) and C5a-desArg ELISA (Dade Behring).

Additionally, at least one ligand is immobilised to said particles or beads, wherein said ligand has a size of about 150 kD or less such as less than 100, 90, 80 or 70 kD. One specific example being avidin having a size of about 66-68 kD. Other examples of ligands are derivatives of avidin, streptavidin, single chain antibodies, scaffolds with antibody like functions, domain antibodies and anticalines and parts thereof. The ligand may be coupled to said particles by any suitable coupling, such as, but not limited to, cyanobromide coupling, glutareldehyde coupling, 2-fluoro-1-methyl-pyridinium toluene 4-sulfonate (FMP) coupling, epichlorohydrin coupling, forming a Schiff's base which can be further reduced by e.g. cyanoborohydride, triazine coupling, or hydrazide coupling. The various means of coupling different ligands such as avidin to polysaccharide particles or beads are well known for one skilled in the art. In the Examples of this invention, avidin has been immobilized to the agarose beads by the use of either epichlorohydrin or by forming a Schiff's base which is further reduced by cyanoborahydride.

In one embodiment, the “ligand” is immobilized to the said particles or beads via a linker.

The porosity of the particles limits the size of the ligand to be used as well as the available binding capacity of the components to be removed. I one aspect of the present invention, the polysaccharide particles or beads are characterized with respect to porosity. The porosity may then be determined by the use of thyroglobulin. The porosity of thyroglobulin (analysed as Kₐᵥ) of said particles being ≧0.29, such as from about 0.29 to about 0.51. The Kₐᵥ (a number between 0 and 1.0) is a measure of how much thyroglobulin enters the bead as opposed to remaining in the void space.

To perform efficiently, in a blood clearance adsorption device, the polysaccharide particles or beads contained in such device, should be such as when tested in a model device and in a model system described in Example 1 and 3, the clearance factor should be at least 0.75, such as at least 0.8, 0.85, 0.9, 0.95 or 1. If the clearance factor is below 0.75 the blood from the mammal to be treated need to pass the device several times to enable sufficient removal of the component to be reduced or removed, and which could otherwise be harmful for the blood as well as the mammal to be treated. For example when using a treatment regime in which the blood is processed three times the efficiency of the device should be such that more than 90% of the component to be removed is removed. The clearance factor is defined above and can be determined by the method described in K. Schindhelm; Artificial Organs 13:21-27 (1989) as well as according to the method in Example 3.

In one embodiment, the polysaccaride particles or beads should be such as when tested in a model device and a model system described in Example 4, the compression factor should be below 10%, such as below 9, 8, 7, 6, 5 or 4% or even lower. The compression factor can be determined by using the method in example 4. If the compression factor is above 10%, the device cannot be used to remove components from whole blood since the device will then collapse due to a high pressure during the use, which is caused by the relatively high viscosity of the blood, which is approximately 4-4.2.

The particles in the adsorption device are polysaccharide particles partly or fully made from agarose or semisynthetic as well as synthetic versions. The polysaccharide particles may be immobilised with avidin and/or steptavidin, such as at least 1 mg avidin and/or streptavidin per ml settled gel. Other examples are 2, 3, 4, 5, 6, 7 or 8 mg avidin/streptavidin per ml settled gel.

The linear flow rate of the adsorption device containing the above described polysaccharide beads (e.g. agarose beads) packed in a column house described in Example 1 should be no less than 2.5 cm/min and still meet the minimal requirement of a clearance factor of 0.77 and a compression factor as defined in Example 4, of less than 10%. Such linear flow rate may be from about 2 to about 5 cm/min such as from about 3 to about 4 cm/min.

In another embodiment, the invention relates to a device as defined above comprising spherical, porous cross-linked particles being immobilised with at least one avidin or streptavidin molecule. Said avidin or streptavidin molecule may be natural as well as semi or synthetic versions thereof.

In another embodiment said avidin molecule is conjugated with at least one biotin molecule as defined above. In yet another embodiment the conjugate above also comprise a targeting agent such as antibodies, such as monoclonal antibodies or vitamins, such as vitamin D, hormones, neurotransmitters, proteins and peptides and parts thereof, synthetic and semisynthetic variants thereof. The antibody can also be an antibody fragment such as F(ab′)₂, F(ab′), 2Fab′, F/ab), genetically engineered hybrids such as a humanised or a chimeric antibody or chemically synthesised peptides. Examples of antibodies are antibodies against different cancers, such as HMFG1, hMN14, trastuzumab (Herceptin®), pertuzumab (Omntarg®), BR96 ibrutomab, Erbitux®, Mylotarg® Zevalin®, LymphoCide, MabCampath®, Oncolym®, vitaxin, Avastin® rituximab and tositumomab.

In one embodiment, the components to be removed or reduced according to this invention should have a size of less than 250 kD such as less than 150 kD or less than 100 kD.

### A Kit Comprising an Adsorption Device

According to one embodiment, the invention relates to a kit for removal of at least one component from blood, such as from whole blood comprising the adsorption device as defined as above and a container (e.g. an infusion bag) comprising at least one pharmaceutically acceptable agent, such as a salt or a solution and at least a first biotin molecule coupled via a linker 1 to a targeting binding moiety. Said container may be any suitable container, such as, but not limited to, a glass bottle or a plastic bag suitable for its intended content as well as adapted to be used together with said adsorption device intended to remove and/or reduce component from blood, such as whole blood.

In one embodiment, the said container is connected to the said adsorption device through a suitable tubing set, and the content of the container is delivered to the adsorption device by the means of a pump or by gravity.

According to another embodiment, the invention relates to a kit for removal of at least one component from blood by the use of an adsorption device comprising polysaccharide particles or beads as defined above and a conjugate, said conjugate comprises, a) a trifunctional cross-linking moiety selected from the group consisting of triaminobenzene, tricarboxybenzene, dicarboxyaniline and diaminobenzoic acid, said trifunctional cross-linking moiety being coupled a) via a linker 1 to a biotin molecule selected from the group consisting of biotin derivatives having essentially the same binding function to avidin or streptavidin as biotin, b) via a linker 2 to a biotin molecule selected from the group consisting of biotin derivatives having essentially the same binding function to avidin or streptavidin as biotin and c) via a linker 3, to a target binding moiety wherein said linkers contains hydrogen bonding atoms.

According to another embodiment the invention relates to a kit for removal from blood of at least one “component” and/or at least one conjugate where the said conjugate is intended to be added to the blood prior to its removal, comprising the adsorption device as defined above and a conjugate comprising a targeting agent/molecule bound via a linker 3 to at least one trifunctional cross-linking moiety selected from the group consisting of triaminobenzene, tricarboxybenzene, dicarboxyaniline and diaminobenzoic acid, said trifunctional cross-linking moiety being coupled a) via a linker 1 to a biotin molecule selected from the group consisting of biotin derivatives having essentially the same binding function to avidin or streptavidin as biotin and b) via a linker 2 to at least one cytotoxic agent.

Said linkers may contain hydrogen bonding atoms, preferably ethers or thioethers, or ionisable groups, preferably carboxylate, sulphonates, amino and ammonium groups to aid in water solubilisation of the biotin moiety, and stability against enzymatic cleavage has been provided by introducing substituents to the biotinamide amine or to an carbon atom adjacent to that amine, such as by the introduction of an alkyl group such as an alpha or beta group, wherein said alkyl group have a length of from C1-C5, being linear or branched and may include carboxyl, carboxy amide or hydroxyl groups (such as alpha or beta aspartyl, aminobutyric acid, serine, theronine, valine etc.). The linkers may provide a spacer length of 1-25 atoms, such as 2, 3, 4, 5, 6, 7, 8, 9, 10, 11, 12, 13, 14, 15, 16, 17, 18, 19, 20, 21, 22, 23 or 24.

According to another embodiment, the invention relates to a kit comprising the adsorption device as defined above and a conjugate as defined above having multiple cytotoxic agents coupled to said conjugate via one or more linkers, wherein said conjugate has an increased cytotoxic effect against the target to which the targeting binding agent can bind.

The kits as defined above may also contain other, agents, such as pharmaceutical agents or drugs as well as solutions, such as heparin and/or citrate solutions.

According to yet another embodiment, the invention relates to the use of said adsorption device or said kit as defined above for the removal and/or reduction of at least one component from blood, such as whole blood. The blood may be from a mammal such as a cow, horse, dog, camel or human being. The blood may in specific embodiments, such as when the adsorption device is an extracorporeal device, be returned to the mammal after at least one component have been reduced and/or removed. In a further aspect of the invention the processed blood is continuously returned during the process through a suitable device such as a blood monitoring machine for extracorporeal treatment.

According to a last embodiment the invention relates to a method for the treatment of a disease or diagnosis of a disease in which the adsorption device or kit as defined above is used. The mammal to be treated may be human as well as other mammals, such as camels, cows, dogs, cats or horsed.

The following examples are intended to illustrate, but not to limit, the invention in any manner, shape, or form, either explicitly or implicitly.

### EXAMPLES

### Example 1

### Preparation of a Mini Adsorption Device

10 batches agarose coupled with avidin were produced and shown in table 1.

The columns were purchased from N E Holm A/S, Birkeröd, Denmark and equipped with 70 μm nets (Sefar 07-70/32) in moulded supports, equipped with bypass tubing rebuilt from Excorim housing. The bed height of the columns was 50 mm. They had a diameter of 16 mm and a bed volume of 10 ml.

10 columns were prepared containing 10 different agarose batches (table 1).

The agarose gel was suspended in buffer PBS-A. The concentration of the agarose was at least 2 volumes of PBS-A per volume of settled gel. 50-60 ml of agarose gel suspension was filled in a syringe connected to the column and the column was filled.

The column was then washed using PBS-A. The washing rate was 6-10 ml/min which was controlled by a Masterflex L/S pump (Buch & Holm, Malmoe, Sweden). The columns were stored at 4° C. until use.

### Example 2

### Measurement of the Particle Size Distribution of Different Batches of Avidin Coupled Agarose Particles

The particle size distribution was determined by microscope using an Olympic MIC-D microscope. The diameter of the particles was correlated to the calibration scale S48 (Graticules, Ltd, UK). 100 particles from each batch were measured and the distribution and mean size was determined (table 1).

### Example 3

### Measurement of Clearance Factor

The functional adsorption properties of the columns prepared in Example 1 were analysed by determination of the clearance factor using the protocol described by K. Schindhelm; Artificial Organs 13:21-27 (1989).

### Materials

Gambro BMM-10 purchased from Excorim, Lund, Sweden, equipped with a blood pump, pressure monitor and air detector or Masterflex L/S pump (Buch & Holm, Malmoe, Sweden) with Easy head II pump head, Testor 512 2000 mbar pressure monitor (Nordtec Instruments AB, Göteborg, Sweden)

Tubing set (Gambro arterial line A-837-A6BU, Sweden), rebuilt for small columns. A 250 ml beaker (diameter 57 mm; solution height about 5 cm) with four fixed spoilers, and a four-wing stirring bar. Biotinylated IgG purchased from Prometic Biosciences Ltd, Cambridge, United Kingdom. High viscosity solution (HVS), having a relative viscosity in the interval 4.0-4.2 (HVS; 80-90 g Dextran (Sigma D3759) per litre of PBSA). The viscosity was determined as the ratio between the viscosity of the solution and the viscosity of water using a 200 μm capillary viscosimeter. The viscosity of the HVS was approximately the same as whole blood.

PBSA: Phosphate-buffered saline, tablets (Sigma P4417) supplemented with 0.05% Sodium azide (Sigma S-2002; 0.5 g/l).

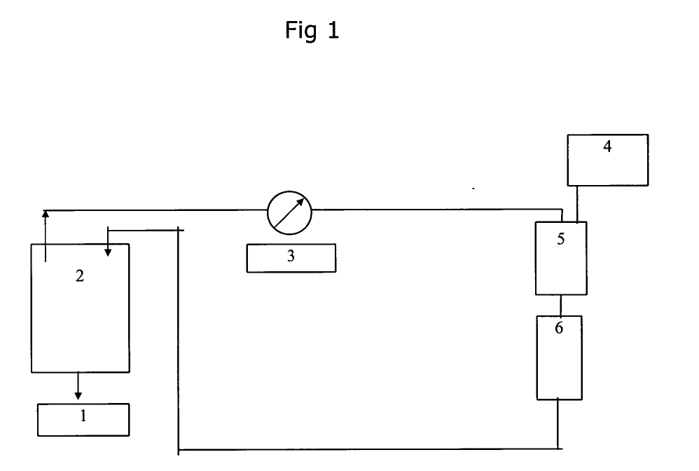

The equipment was set up as shown in FIG. 1 with re-circulation of the biotin-IgG/HVS through the column at 6.25 ml/min. The 250 ml beaker had a stirring speed of 6 (IKA Labortechnik, Tumro Medlab, Sweden).

The column was primed with 60 ml PBS-A followed by 60 ml HVS. During the priming the flow rate was adjusted to 6.25 ml/min. The column was then set in bypass-mode and the tubing was emptied. The reservoir was weight prior to and after the run.

12.5 mg of the biotinylated IgG was added to 125 ml HVS in the reservoir. Before start the system was primed 5 minutes with the biotin IgG solution via the by-pass tubing. Two samples were taken after 4 minutes. At start the flow was opened to the column. During the recirculation through the column, samples were collected after 2, 4, 6, 8, 10, 14, 18, 22, 26, 30, 34, 38, 42, 46, 50, 54, 58, 62, 66, 70 and 74 minutes after start. During the adsorption of the biotinylated IgG to the column the column was visually inspected for compression of the column.

The column was set in by-pass mode and additional 12.5 mg of biotinylated IgG was added to the reservoir and re-circulated for 5 minutes. Two samples were taken after 4 minutes. The flow was opened to the column. During re-circulation through the column, samples were collected at the same intervals as mentioned above. Samples were taken both from the reservoir and the outlet.

The sample volume was 200 μl.

The pressure was registered during the process.

The amount of biotinylated IgG was measured by ELISA, well known for a person skilled in the art, and the clearance factor was calculated.

The clearance factor was calculated as the slope obtained from the regression analysis of ln(% biotin-IgG) against the number of processed re-circulation volumes.

Volume to be used for calculation was the volume of HVS in the reservoir minus the volume in the tubing set run in bypass.

The lower limit of 95% significance confidential interval for the slope was also calculated.

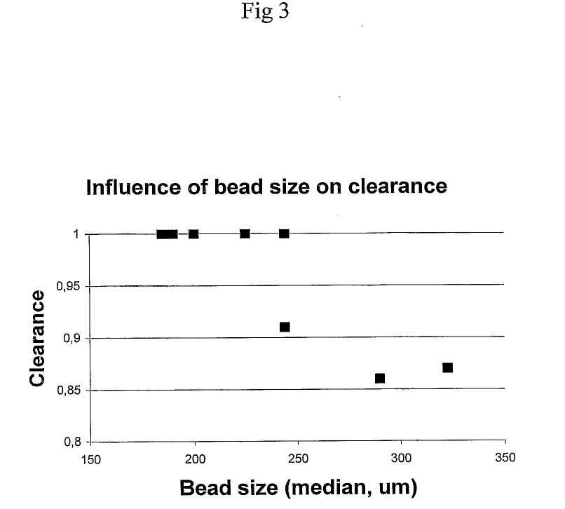

The results are shown in table 2 and FIG. 3.

### Example 4

### Measurement of the Compression Factor

The compression factor was determined during the analysis of the flow/pressure test.

The same materials and system was used as in example 3.

The system was set up for re-circulation of the buffer through the system. The system was primed with 6.5 ml/min with the PBSA buffer. The inlet and outlet tubing was at the same vertical level.

The pump setting was increased in intervals of 50 ml/min, starting at 100 ml/min (corresponding to 6.5 ml/min flow rate), until a pressure of 250 mm Hg was reached. The pressure and flow rate was registered at each pump setting, which was approximately 5 minutes. The volume of buffer was collected during 2 minutes (5-7 minutes). At the end of each flow rate tested, the column was visually inspected for compression of the adsorbent material. And the bed height registered.

The flow rate was calculated by dividing the collected buffer volume with the collection time.

The results are shown in table 3 and table 4.

### Example 5

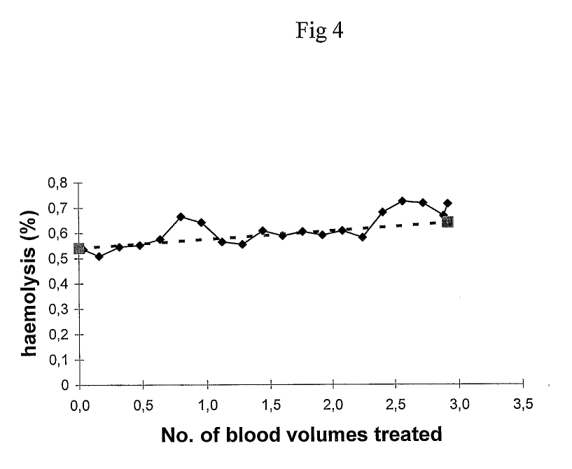

Haemolysis was monitored during adsorption. No haemolysis could be detected by the naked eye. When the plasma haemoglobin levels were analysed by the colourometric assay a slight increase was detected. During adsorption the haemolysis increased from 0.54% to 0.71% (maximal during treatment=0.72). Therefore, the net increase was 0.17%. An untreated standby blood sample increased to 0.64% during the same time period; net increase 0.10%. The variation that was seen results probably from inaccuracy of the assay. (FIG. 4) (Filled line: haemolysis during adsorption, dotted line: increase in untreated blood).

### Example 6

### Conjugation of an Antibody to a Trifunctional Cross-Linking Unit

The monoclonal antibodies hMN14 (CEA-Cide®, Immunomedics), BR96 (Seattle Genetics), trastuzumab (Herceptin®, Roche) and rituximab (Mabthera®, Roche) were conjugated with the trifunctional chelator 1033 (MitraTag®, Mitra Medical AB, Lund, Sweden), carrying a DOTA moiety and a biotin moiety (Wilbur et al., Bioconjugate Chem, 2002; 13:1079-1092). Prior to conjugation were the monoclonal antibodies dialysed against HEPES, 1 mM DTPA buffer (pH8.5). Conjugation was performed by adding 80 μg of MitraTag® per mg monoclonal antibody and incubated for 2 hours at room temperature and over night at +4° C. After conjugation the conjugate was transferred to a storage medium containing 0.25 M ammonium acetate (pH5.3). The number of 1033 molecules per monoclonal antibody was determined by the HABA photometric method described by Green, Biochem J 1965; 94:23c-24c.

TABLE 1

| | Batch | | | | | | | | | |
|---|---|---|---|---|---|---|---|---|---|---|
| | 1 | 2 | 3 | 4 | 5 | 6 | 7 | 8 | 9 | 10 |
| mg Avidin/ml gel | 5-7 | 5-7 | 5-7 | 5-7 | 5-7 | 5 | 2 | 5 | 5-7 | 5 |
| mean | 198 | 199 | 201 | 225 | 206.4 | 184.6 | 246 | 253 | 326.3 | 285.9 |
| median | 189 | 200 | 190 | 225 | 200 | 185 | 244 | 244 | 322.5 | 290 |
| % in range 180-250 μm | 50 | 52 | 58 | 70 | 46 | 58 | 63 | 51 | 0 | 18 |
| % in range 150-300 μm | 99.3 | 98.7 | 99 | 98 | 100 | 100 | 94 | 95 | 36 | 76 |
| Min | 133 | 140 | 110 | 135 | 150 | 150 | 178 | 178 | 275 | 200 |
| Max | 311 | 325 | 333 | 310 | 300 | 285 | 333 | 322 | 400 | 360 |

TABLE 2

| | 1 | 2 | 3 | 4 | 5 | 6 | 7 | 8 | 9 | 10 |
|---|---|---|---|---|---|---|---|---|---|---|
| Run 1 | | | | | | | | | | |
| Clearance | 1.07 | 1.06 | 1.02 | 1.14 | 1.10 | 1.29 | 0.91 | 1.21 | 0.87 | 0.86 |
| 95% | 1.00-1.13 | 1.00-1.11 | 0.94-1.10 | 1.08-1.21 | 1.00-1.20 | 1.22-1.36 | 0.86-0.96 | 1.17-1.25 | 0.82-0.92 | 0.80-0.91 |
| conf. int. | | | | | | | | | | |
| corr. coeff. | 0.98 | 0.99 | 0.98 | 0.98 | 0.99 | 0.99 | 0.99 | 1.00 | 0.99 | 0.99 |
| Run 2 | | | | | | | | | | |
| clearance | 0.75 | 0.77 | 0.74 | 0.85 | 0.87 | 1.08 | 0.61 | 1.03 | 0.54 | 0.59 |
| 95% | 0.66-0.84 | 0.74-0.81 | 0.67-0.82 | 0.82-0.87 | 0.83-0.91 | 0.98-1.18 | 0.56-0.67 | 0.98-1.07 | 0.50-0.58 | 0.53-0.65 |
| conf. int. | | | | | | | | | | |
| corr. coeff. | 0.94 | 0.99 | 0.96 | 1.00 | 0.99 | 0.97 | 0.97 | 0.99 | 0.98 | 0.97 |

TABLE 3

| Pump setting | 1 | 2 | 3 | 4 | 5 | 6 | 7 | 8 | 9 | 10 |
|---|---|---|---|---|---|---|---|---|---|---|
| | Flow rate | | | | | | | | | |
| | (ml/min) | | | | | | | | | |
| 1.0 | 6.3 | 6.3 | 6.25 | 6.25 | 6.3 | 6.3 | 6.3 | 6.3 | 6.3 | 6.3 |
| 1.5 | 8.5 | 7.8 | 7.6 | 8.5 | 7.9 | 8.4 | 7.9 | 8.0 | 8.0 | 8.0 |
| 2.0 | 11.0 | 10.5 | 10.5 | 11.0 | 10.5 | 11.1 | 10.5 | 10.5 | 10.5 | 10.5 |
| 2.5 | 14.3 | 13.0 | 13.2 | 13.8 | 13.0 | 14.0 | 13.1 | 13.4 | 13.5 | 13.5 |
| 3.0 | 17.0 | 15.5 | 15.9 | 16.5 | 15.5 | 16.6 | 16 | 16.0 | 16.0 | 16.0 |
| | Pressure | | | | | | | | | |
| | (mmHg) | | | | | | | | | |
| 1.0 | 65 | 70 | 60 | 80 | 85 | 75 | 70 | 50 | 60 | 50 |
| 1.5 | 80 | 80 | 75 | 100 | 95 | 90 | 80 | 60 | 70 | 60 |
| 2.0 | 105 | 100 | 98 | 125 | 130 | 120 | 110 | 75 | 90 | 80 |
| 2.5 | 150 | 130 | 125 | 160 | 160 | 150 | 145 | 100 | 110 | 100 |
| 3.0 | 185 | 170 | 160 | 190 | 200 | 175 | 175 | 110 | 130 | 125 |

TABLE 4

| | Height of adsorbent bed | | | | | | | | | |
|---|---|---|---|---|---|---|---|---|---|---|
| | (mm) | | | | | | | | | |
| Pump setting | 1 | 2 | 3 | 4 | 5 | 6 | 7 | 8 | 9 | 10 |
| 1.0 | 50.0 | 50.0 | 50.0 | 50.0 | 49.5 | 50.0 | 50.0 | 50.0 | 50 | 50 |
| 1.5 | 50.0 | 50.0 | 50.0 | 50.0 | 49.0 | 50.0 | 50.0 | 49.5 | 50 | 50 |
| 2.0 | 49.5 | 50.0 | 50.0 | 49.8 | 48.0 | 50.0 | 50.0 | 49.5 | 50 | 50 |
| 2.5 | 48.0 | 49.0 | 50.0 | 49.0 | 47.0 | 50.0 | 49.5 | 49.0 | 50 | 50 |
| 3.0 | 47.0 | 47.0 | 49.0 | 48.0 | 46.0 | 50.0 | 49.0 | 49.0 | 49.5 | 49.5 |

1. An adsorption device for removal and/or reduction of at least one component from blood comprising porous polysaccharide particles wherein
a) the particles have an average size from about 210±50 μm,
b) at least 30% of the particles have a size from about 180 to about 240 μm,
c) at least 70% of the particles have a size from about 150 to about 300 μm and
said particles having at least one immobilised ligand, wherein said immobilised ligand has a size of about 150 kD or less.

2. The adsorption device according to claim 1, wherein said particles are beads.

3. The adsorption device according to claim 1, wherein said polysaccharide particles are cross-linked.

4.-11. (canceled)

12. The adsorption device according to claim 1, wherein said immobilised ligand is selected from the group consisting of avidin and streptavidin.

13. The adsorption device according to claim 1, wherein said immobilised ligand is selected from the group consisting of immunoglobulins, Fab-fragments, single chain antibodies, scaffolds with antibody like functions based on lipocalin, scaffolds with antibody like functions based on protein A, domain antibodies and conjugates of domain antibodies.

14. The adsorption device according to claim 13, wherein scaffolds with antibody like functions are selected from the group of lipocalins, anticalins and affibodies

15.-20. (canceled)

21. The adsorption device according to claim 1, wherein said particles have a porosity value (Kav) of ≧0.29.

22. (canceled)

23. The adsorption device according to claim 1, having a clearance factor of at least 0.75.

24.-28. (canceled)

29. The adsorption device according to claim 1, having a compression factor below 10%.

30.-35. (canceled)

36. The adsorption device according to claim 1, wherein the particles comprises agarose as one of the components.

37. The adsorption device according to claim 36, wherein the particles are agarose particles or beads.

38. The adsorption device according to claim 37, wherein the particles are agarose beads.

39. The adsorption device according to claim 1, wherein the particles are immobilised with at least 1 mg avidin or streptavidin/ml settled agarose gel.

40.-45. (canceled)

46. The adsorption device according to claim 1, wherein the linear flow rate is from about 2 to about 5 cm/min

47.-48. (canceled)

49. The adsorptions device according to claim 12, wherein said adsorption device comprises a target binding moiety linked to a biotin moiety or biotin derivative and where said biotin/biotin derivative is linked to the immobilized avidin or streptavidin.

50. The adsorptions device according to claim 49, where said biotin/biotin derivative is linked to the target binding moiety through a linker.

51. The adsorptions device according to claim 12, wherein said adsorption device comprises a conjugate,
said conjugate comprises: a) a trifunctional cross-linking moiety selected from the group consisting of triaminobenzene, tricarboxybenzene, dicarboxyaniline and diaminobenzoic acid, said trifunctional cross-linking moiety being coupled
a) via a linker 1 to a biotin molecule selected from the group consisting of biotin derivatives having essentially the same binding function to avidin or streptavidin as biotin,
b) via a linker 2 to a biotin molecule selected from the group consisting of biotin derivatives having essentially the same binding function to avidin or streptavidin as biotin and
c) via a linker 3, to a targeting binding moiety wherein said linkers contain hydrogen bonding atoms.

52. The adsorptions device according to claim 51, wherein said adsorption device comprises a conjugate where the linker 3 is omitted.

53. A kit for the removal of at least one component from blood comprising:
a) the adsorption device according to claim 12 and
b) a container comprising at least a pharmaceutically acceptable agent and at least a first biotin molecule coupled via a linker 1 to a targeting binding moiety.

54. A kit for the removal of at least one component from blood according to claim 53 where linker 1 is omitted.

55. A kit for removal of component from blood comprising
a) the adsorption device according to claim 12 and
b) a conjugate, said conjugate comprises, a) a trifunctional cross-linking moiety selected from the group consisting of triaminobenzene, tricarboxybenzene, dicarboxyaniline and diaminobenzoic acid, said trifunctional cross-linking moiety being coupled
i) via a linker 1 to a biotin molecule selected from the group consisting of biotin derivatives having essentially the same binding function to avidin or streptavidin as biotin,
ii) via a linker 2 to a biotin molecule selected from the group consisting of biotin derivatives having essentially the same binding function to avidin or streptavidin as biotin and
iii) via a linker 3, to a target binding moiety wherein said linkers contain hydrogen bonding atoms.

56. A kit for removal of component from blood comprising
A) the adsorption device according to claim 12 and
b) a conjugate, comprising a targeting agent/molecule bound via a linker 3 to at least one trifunctional cross-linking moiety selected from the group consisting of triaminobenzene, tricarboxybenzene, dicarboxyaniline and diaminobenzoic acid, said trifunctional cross-linking moiety being coupled
i) via a linker 1 to a biotin molecule selected from the group consisting of biotin derivatives having essentially the same binding function to avidin or streptavidin as biotin,
ii) via a linker 2 to at least one cytotoxic agent.

57. (canceled)

58. The kit according to claim 53 wherein the container is an infusion bag.

59. A method of removing a component from blood comprising using the adsorption device according to claim 1.

60. A method for the treatment of a disease or diagnosis of a disease comprising the step of removing a component from blood by using the adsorption device according to claim 1.
